(19) United States
(12) Patent Application Publication
(10) Pub. No.: US 2003/0026859 A1
(43) Pub. Date: Feb. 6, 2003

(54) Antiviral composition derived from Allium cepa and therapeutic use thereof

(76) Inventors: Adolfo Goren, Condnha (AR); Walter Franklin Goldman, Houston, TX (US); Zila Trainin, Rahovoe (IL); Simon Raul Goldman, Cardoba (AR)

Correspondence Address: Pillsbury Winthrop LLP, Intellectual Property Group, 1600 Tysons Boulevard, McLean, VA, 22102, US

(21) Appl. No.: 10/076,247

(22) Filed: Feb. 14, 2002

Related U.S. Application Data
(62) Division of application No. 09/631,284, filed on Aug. 3, 2000, now Pat. No. 6,340,483.

(51) Int. Cl. A61K 35/78

(52) U.S. Cl. 424/754

(57) ABSTRACT

Novel medicinal extracts derived from Allium species, preferably Allium cepa are provided. These extracts have broad medicinal properties, especially for treatment of ADS and other viral infections.

### FIELD OF THE INVENTION

The invention relates to a novel plant extract and therapeutic use thereof. More particularly, the present invention relates to the use of a novel plant extract for treating AIDS and other viral infections.

### BACKGROUND OF THE INVENTION

The use of plant derived compositions as therapeutic agents has been known for thousands of years. In particular, the Chinese are known for their herbal therapies. Recently, there has been a resurgence in the use of natural and plant-derived materials that supposedly possess therapeutic activity and promote general well being. For example, many persons now take St. John's Wort, purportedly to alleviate depression and promote general well being. Also, Ginkgo Biloba, purportedly to enhance memory, is widely used now. Further, SAMe, a yeast-derived material which purportedly is useful for treatment of osteoarthritis and alleviation of depression, is in wide use.

In particular, the use of materials derived from a plant of the Allium family, especially Allium sativum (“garlic”) has been reported in the literature. For example, several patents by Tatarintsev et al report the use of ajoene compound, derived from the garlic plant, for treating a variety of ailments including AIDS, inflammation, arthritis, transplant, infection, autoimmune diseases such as lupus, tuberculosis, tumors, and other relates diseases. (See U.S. Pat. Nos. 5,856,363; 5,863,955; 5,948,821; and 5,932,621.)

Also, Hibi, U.S. Pat. No. 5,612,077, describes an ajoene-containing extract from garlic for use in treating arteriosclerosis, tuberculosis, and bronchitis. Further, Tsuei, U.S. Pat. No. 4,795,636; Seebeck, U.S. Pat. No. 2,642,374; and Spinka et al, U.S. Pat. No. 2,618,561, describe garlic extracts as medicinal agents.

Further, the isolation of compounds from plant materials, including Allium that inhibit apoptosis has been reported. See especially, U.S. Pat. Nos 5,567,425; 5,759,548; 4,986,985; 5,620,885; 5,624,672; 5,635,186; and 5,635,187, by Bathurst et al, all of which are incorporated by reference in their entirety herein.

The foregoing is only exemplary of plant-derived materials reported to possess medicinal properties. However, notwithstanding the large number of plant-derived materials reported to possess therapeutic properties, there still exists a need for novel plant extracts and therapeutic use thereof as such therapies may be safer and more cost effective than traditional medical treatments.

### OBJECTS OF THE INVENTION

It is an object of the invention to provide a novel plant extract derived from a plant species of the family Alliaceae (also known as Lilliaceae or Amaryllidaceae), preferably of the genus Allium, with the proviso that said Allium is not garlic (Allium sativum), having medicinal properties.

It is a more specific object of the invention to provide novel plant extracts derived from an Allium species selected from the group consisting of Allium cepa, Allium ampeloprasum (“leek”), Allium fistulosa (Japanese bunching onion, scallion or Welsh onion), or Allium schoenoprasum (“chives”) having medicinal properties.

It is an even more specific object of the invention to provide a novel plant extract derived from Allium cepa, preferably Allium cepa var. Ancasti or Southporth white glove (SWG) having medicinal properties.

It is a more specific object of the invention to provide a novel method of treating or preventing a retroviral infection such as HIV-1 or HIV-2 or AIDS by administering a plant extract derived from an Allium species other than Allium sativum, preferably Allium cepa, Allium fistulosa, Allium ampeloprasum or Allium schoenoprasum, and most preferably Allium cepa.

It is a more specific object of the invention to treat wasting syndrome, especially associated with AIDS, and/or to lengthen the latency period of HIV infection, and/or to delay the latent phase of ADS, and/or to ameliorate or eliminate the clinical symptoms associated with AIDS such as intestinal problems, diarrhea, neurological impairment and paresthesia by administering a medicinal extract derived from an Allium species other than Allium sativum, preferably Allium cepa, Allium fistulosa, Allium ampeloprasum or Allium schoenoprasum, and more preferably Allium cepa.

It is another specific object of the invention to inhibit or treat microbial infection, e.g., fungal, yeast or Candidiasis infection in a subject in need of such inhibition or treatment, comprising administering a medicinal extract derived from an Allium species other than A. sativum, preferably Allium cepa, Allium fistulosa, Allium ampeloprasum or Allium schoenoprasum and most preferably Allium cepa.

It is another specific object of the invention to modulate the immune system of a subject in need of such treatment, by administering a medicinal extract derived from an Allium species other than Allium sativum, preferably Allium cepa.

It is an object of the invention to provide a novel method of treating or preventing viral infection by administering a medicinal extract derived from an Allium species other than Allium sativum.

It is a more specific object of the invention to provide a novel method of treating or preventing viral infection by administering a medicinal extract derived from an Allium species selected from the group consisting of Allium cepa, Allium ampeloprasum, Allium fistulosa, and Allium schoenoprasum.

It is an even more specific object of the invention to provide a novel method of treating or preventing viral infection by administering a medicinal extract derived from Allium cepa, preferably the variety Ancasti or Southporth white glove.

It is another object of the invention to immunostimulate the immune system of a subject in need of such treatment by administering a medicinal extract derived from an Allium species other than sativum, preferably Allium cepa, Allium fistulosa, Allium schoenoprasum or Allium ampeloprasum and more preferably Allium cepa.

It is another object of the invention to enhance T cell function and/or T cell proliferation, and/or T-cell differentiation, by administering a medicinal extract derived from an Allium species other than Allium sativum, preferably Allium cepa, Allium fistulosa, Allium schoenoprasum or Allium ampeloprasum, most preferably Allium cepa.

It is another specific object of the invention to identify and isolate the active constituent or constituents comprised in the Allium extract disclosed herein having antimicrobial, antifungal, antiviral, immunomodulatory, T cell function or proliferation inducing, and/or immunostimulatory activity.

It is another specific object of the invention to promote weight gain in a subject in need of such treatment by administering an effective amount of a medicinal extract derived from an Allium selected from Allium cepa, Allium fistulosa, Allium schoenoprasum or Allium ampeloprasum, most preferably Allium cepa.

It is another object of the invention to treat animal microbial infections, especially viral infections such as distemper or parvovirus or bacterial infections such as psittacosis, by administering a medicinal extract derived from Allium cepa.

It is another object of the invention to provide a method for producing a medicinal extract from Allium species selected from Allium cepa, Allium fistulosa, Allium schoenoprasum or Allium ampeloprasum.

It is another object of the invention to provide a novel medicinal extract derived from Allium cepa, Allium fistulosa, Allium schoenoprasum, or Allium ampeloprasum, preferably orally administrable.

It is another specific object of the invention to provide a novel regimen for treating AIDS that includes a dietary regimen, no administration of conventional AIDS medications such as protease inhibitors, anti-retrovirals, or other chemotherapeutics causing multiple side effects (other than antibiotics) and administration of a medicament derived from a plant of the genus Allium, preferably Allium cepa.

### DETAILED DESCRIPTION OF THE INVENTION

The present invention is based on the discovery that plants of the family Alliaceae, specifically those of the genus Allium, including Allium cepa, and related species such as Allium fistulosa, Allium schoenoprasum, and Allium ampeloprasum, can be used to obtain plant extracts having significant medicinal properties. In particular, it has been surprisingly discovered that extracts derived from Allium species, preferably Allium cepa, have wide ranging medicinal properties, including antiviral activity, antimicrobial activity (antifungal, antibacterial), immunomodulating activity, immunostimulating activity, T-cell function and/or T-cell proliferation and/or T-cell differentiation enhancing activity, and weight gain promoting activity.

This discovery is surprising in the fact that onions, while anecdotally reported to possess some medicinal properties, e.g., inhibition of thrombocyte aggregation and lipid and blood pressure lowering activity, have never been suggested to possess the wide ranging medicinal activities discovered by the present inventors, especially regarding AIDS.

Therefore, the subject invention provides novel medicinal extracts and methods of use thereof, wherein such medicinal extracts are derived from a plant of the family Alliaceae (also known as Liliaceae or Amaryllidaceae), preferably Allium but excluding Allium sativum, and preferably Allium cepa, Allium fistulosa, Allium schoenoprasum or Allium ampeloprasum. Most preferably, the novel medicinal extracts of the present invention will be derived from onion (Allium cepa L.).

The onion, Allium cepa L. (2n=16 diploid), is a monocotyledon of the family Alliaceae which is by far the most economically important bulb vegetable. It is thought that the onion originated in Persia (Nonnecke, Ill., “Vegetable Production”, Van Nostrand Reinhold, N.Y. (1989)) cultivated by the Egyptians, Greeks and Romans and brought to North America by the Spaniards. The onion has long been considered to possess medicinal value (Nonnecke (Id.)).

Allium cepa is divided into three main groups.

1. The common onion—bulbs are formed as single plants, and the inflorescence does not form bulbets (Nonnecke, (Id.)1989). The bulk of the onion cultivars belong to this group, which is the most important commercially (Nonnecke, (Id.)1989). It is propagated in the main from true seeds. Within this group exist extremes of bulb shapes (present-day cultivars include the Sweet Spanish, Bermuda, and globe onions), dry scale color (white, yellow, and red predominating), pungency (ranging from mild and sweet to pungent), and other characteristics (Nonnecke, (Id.)1989). When harvested early they produce spring or bunching onions (Lorenz, Onion, In “The Software Toolworks Multimedia Encyclopedia”, Version 1.5, Grolier, Inc. (1992)). The onion plant is potentially a biennial, producing large bulbs the first year and seed the next (Lorenz, (Id.)1992). Plants may be grown from seed, as transplants of seedlings, or as small bulbs (sets) produced from thickly planted seed; when replanted, these bulbs reach maturity rapidly (Lorenz, (Id.)1992). Mature onions are usually dried before marketing (Lorenz, (Id.)1992).

2. The aggregatum group—characterized by many lateral bulbs or shoots, inflorescences lacking bulbets, sterile seed production, and propagation by vegetative means (Nonnecke, (Id.)1989). This group includes the potato onion or multiplier onion, ever-ready onions, and shallots (shallots are sometimes called scallions, a source of confusion because A. fistulosum is also called scallions) (Nonnecke, (Id.)1989).

3. The proliferous (proliferum) group—in this group, ground bulbs are sometimes poorly developed, the inflorescence bears bulbets, true seed is usually lacking, and therefore reproduction is by vegetative inflorescence and bulbets (Nonnecke, (Id.)1989). These are not commercially cultivated; they are used almost exclusively for home gardening (Nonnecke, (Id.)1989). The most common names for this group are tree onions, top-set onions, and Egyptian onions (Nonnecke, (Id.)1989).

The common onion (1. above), is a herbaceous biennial normally producing seed stems in the form of an umbel consisting of florets (Nonnecke, (Id.)1989). The swollen base of the stem forms a bulb made up of numerous fleshy leaves (Nonnecke, (Id.)1989). Short day length varieties are adapted for use in areas south of 30° N (Nonnecke, (Id.)1989). Medium day length varieties are adapted to mild areas from 30° N to about 38° N, in areas where comparatively mild winters occur: south to central California, Georgia and mid-Atlantic states (Nonnecke, (Id.)1989). The long day onions are adapted for spring seeding or transplanting in sites north of 38° N (Nonnecke, (Id.)1989). The longer day length compensates for the shortness of the growing season by providing longer periods of photosynthetic activity (Nonnecke, (Id.)1989). It is absolutely critical to choose the appropriate cultivar for the appropriate environment; when short-day cultivars are grown in a long-day growing area or vice versa, the onion will not perform as expected Nonnecke, (Id.)1989).

Onions for dehydration are white and have high soluble solids (Nonnecke, (Id.)1989). They are grown mostly in California where the long growing season permits the right balance of solids and pungency desired for the dehydrated product (Nonnecke, (Id.) 1989). The American market classifies onions according to maturation time (Nonnecke, (Id.)1989). The Bermuda Granex, a grano type that is mild and flat top shaped, is an early-maturing onion with little or no storage life (Nonnecke, (Id.)1989). The late crop onions, mostly globe shaped with yellow, white or red scales are mild or pungent depending on cultivar and environment, and usually store well over long periods (Nonnecke, (Id.)1989). The bulk of the seed is produced in an environment conducive to good seed set, such as California (Nonnecke, (Id.)1989).

In general, the medicament of the present invention which is derived from Allium cepa, Allium fistulosa, Allium schoenoprasum or Allium ampeloprasum, and preferably Allium cepa, more preferably type Ancasti and Southporth white glove, is produced by dehydration of an Allium plant material other than Allium Sativum by one or more heating steps, typically effected at about 80° C./110° C. or higher, i.e., after an initial washing procedure, removal of non-viable materials and removal of other impurities such as stones, wires, etc., and processing of the dehydrated material into fine particles or granules, i.e., roughly having the consistency of talcum powder or confectionary sugar, such that the average particle size ranges from about 1 to 1,400 microns and, more preferably, ranges from about <250 to about 850 microns. This granulation or particulation procedure can be effected or known industrial devices for effecting granulation, or in particular by use of high speed cutters such as those available from Moulinex or other suppliers. Such granulation will preferably be effected at low humidity, e.g., ≦5%-7% humidity, in order to prevent aggregation or clumping of particles or granules during processing. It is hypothesized by the present inventors that the particulate size of the subject medicament may enhance its medicament properties, perhaps because of increased surface area. Alternatively, it is speculated that particles of such size may facilitate absorption or uptake of the active constituent by specific cells, e.g., immune cells, and thereby potentiate its effect on the immune system.

Preferably, the utilized procedure for particulating the processed dehydrated Allium, preferably Allium cepa, material will result in most (greater than 95%) of the particles or granules in the resultant composition having an average size ranging from 1 to 1,400 microns, more preferably most of the particles will have an average particle size in the range of ≦10 microns to about 850 microns, and most preferably most of the particles will have an average size within the range of 36 to 500 microns.

A preferred procedure for producing an Allium medicinal extract according to the invention comprises the following steps. This process is exemplary of the processes that can be used to produce the novel medicinal extract of the invention. Indeed, it is anticipated that the ordinary routineer will be able to modify this procedure without adverse effects, e.g., to reduce costs and enhance efficacy.

In the exemplary process, an Allium plant material, preferably Allium cepa, Allium fistulosa, Allium ampeloprasum or Allium schoenoprasum, preferably Allium cepa, including at least the bulb portion of the plant, is obtained. In a preferred embodiment, the selected Allium cepa material will comprise Allium cepa variety ancasti or Southporth white glove. However, this is not essential to the invention, and other Allium cepa varieties may alternatively be utilized. Preferably, the selected Allium cepa or above-identified Allium species will be grown in the absence of herbicides, insecticides and other agrochemicals such as organic fertilizers. However, this also is not essential to the invention.

This Allium plant material, which includes especially the bulb portion of the plant, may be stored prior to medicament preparation, preferably under cold conditions, typically about 10° C. at 70% humidity, or about 5-15° C. at 60-80% humidity. For example, the Allium plant material may be stored in wooden boxes (bines) for prolonged period, e.g., at least 30 days, and up to about 4 months. If stored for longer periods, the active constituents may lose their activity, e.g., because of natural decomposition of the onion over time.

Thereafter, the Allium plant material containing the bulb portion, optionally after it has been stored under appropriate conditions undergoes a quality evaluation. This material may also be classified based on size of plant materials.

Thereafter, the plant material is subjected to one or more washing procedures. Preferably, this washing will be effected under abrasive conditions on a conveyor belt which comprises use of an acidic aqueous washing solution, preferably a chlorinated aqueous solution. In a preferred embodiment, the washing solution will contain about 100 to 120 parts per million of chlorine. Preferably, no other chemical additives will be utilized during the washing procedure to eliminate fungus or bacteria. As noted, the washing is effected under abrasive conditions, i.e., the bulbs are also treated by abrasion, e.g., by a brushing procedure, at this time to remove the outer layers. Thereafter, one or more additional washing steps are then preferably effected, again using a chlorine-containing aqueous washing solution, preferably a cold chlorine-containing aqueous solution.

More specifically, the subject medicament is produced using an industrial process that complies with the F.D.A. regulations of the U.S.A. under the quality ISO 9000 norms. Preferably, the bulb recollection is industrial, not manual. After collection, the bulbs are preferably stored in wooden boxes of 1m×1m×1m called bines. To maintain stability, such bulbs are stored in chambers at an average of 10° C. at 70% humidity (5-15° C. and 60-80% humidity.) The normal storage does range between 30 and 120 days. This is followed by a quality evaluation (defects, solid material content, etc.). The optionally sorted bulbs are then sent to the elaboration plant.

The vegetal material is loaded in a large recipient or feeder to transport the material by a conveyor belt to start the first step in the process. In one embodiment the conveyor belt is made of a sequence of cylinders with brushes upon which the vegetal material rolls. Thereafter, “washing by abrasion” is effected with cold chlorinated water (e.g., about 7 to 15° C.). The chlorine concentration preferably ranges from 100 to 120 parts per million. No additive is used to eliminate fungus and bacteria. The bulbs are also brushed to eliminate the outer layers. This process of washing by abrasion is repeated a second time under identical conditions.

A belt is then used to transport the bulbs to the place where the material is sorted, e.g., manually, by discarding the non-viable one (e.g., green non-comestible or rotten material.) Thereafter, the material goes through a process of decantation eliminating foreign bodies such as stones, wires, etc. The bulbs are then cut into thin slices (e.g. 2-7 mm), preferably about 2-4 mm., e.g., by use of a cutting machine, to help the process of selection and dehydration. This is followed by the process of dehydration. For example, in one embodiment, the material is transported using another belt, and the transported material is preferably then heated, e.g. in an oven of dry continuous heat, and preferably first at 80-100° C. for 45 minutes, followed by 90-110° C. for another 45 minutes, and finally dehydration is completed at 80-100° C. for 30 minutes. This final material (called virgin) contain 5-7% humidity. However, it is anticipated that the heating process may be varied without adverse effects.

The virgin is kept in sealed bags at 20 to 25° C., preferably under dark conditions, until it will be needed. When needed for use, the “virgin” material is then processed with another machine that strips the outer layers and leaves the pulp by pneumatic separation. In this way, the slices obtained are of pulp material. This pulp is then chopped into flakes, which preferably are kept sealed at 18-25° C., away from sunlight.

After drying, the plant material, which consists of the pulp (and optionally outer layers) is then loaded into an industrial processing device, e.g., high speed cutters. Other option is the use of devices such as “Moulinex”, or from other suppliers, that processes this material into fine particles or granules.

Preferably this granulation is effected at very low humidity, i.e., maximum of about 5.5% humidity to avoid clumping, in order to provide granulates having an average particle size ranging from about 1 to 1,400 microns, more preferably from about ≦36 microns to 850 microns. The dried particles will typically comprise at most 5.5% water, and preferably less. In an exemplary composition produced according to the invention from Allium cepa, about 42.9% of particles were smaller than 250 microns, 56.9% were less than 355 microns, 74.7% were less than 500 microns, and 2f.7% were between 500-850 microns, with 22.1% ranging from 106-250 microns, 6.8% ranging from 75-106 microns, 10.8% ranging in size from 36-75 microns, and 3.2% being <36 microns. Thus, the significant majority of particles are less than 500 microns, with most ranging from <36 to 850 microns.

The dried particulate material may be immediately used as a medicament or it may be stored for a prolonged time prior to usage. This material preferably will be stored under cold (typically about 18 to 25° C.), dark conditions, for up to a year or even longer. This will prevent degradation of active constituents, e.g., by oxidation or sunlight.

It is believed that the subject treatment process which essentially comprises acidic washing steps, heated dehydration, and granulation into very fine particles having the consistency of a fine powder, e.g., on the order of talcum powder, results in an extract having a substantially unchanged chemical composition from the original Allium material, except for the removal of water, scent, and some other volatile acids.

However, the present inventors do not rule out the possibility that the washing, dehydration process and the heating steps used in the subject matter may be facilitating one or more reactions that result in the formation of one or more medicament compounds that are not endogenously present in Allium or which were present in lower concentrations, may provide for the release of larger amounts of active constituents. With respect thereto, the subject inventors are uncertain as to what are the exact active constituent or constituents that are contained in the subject medicament composition.

However, it has been reported that Allium cepa comprises the compounds listed below.

1-(F)-β-FRUCTOSYL-SUCROSE

1-(METILSULFINYL)-PROPYL-METHYL-DISULFIDE

1-METHYLDITHIO-PROPANE

1-METHYLTRITHIO-PROPANE

1-0-CAFFEOYL-β-D-GLUCOSE

1-0-FERULOYL-β-D-GLUCOSE

1-0-P-COUMAROYL-β-D-GLUCOSE

1-PROPYLTRlTHOPROPANE

2,3-DIMETHYL-(D,L)-BUTANE-CIS-1-CIS-D-S,S′-DIOXIDE

2,3-DIMETHYL-5,6-DITHIA-BICYCLO-(2,2,1)-HEXANE-5-OXIDE

2,3-DIMETHYLTHIOPHENE

2,4-DIMETHYLTHIOPHENE

2,5-DIMETHYLTHIOPHENE

24-METHYLENE-CYCLOARTENOL

28-ISOFUCOSTEROL

2-METHYL-BUT-2-EN-1-AL

2-METHYL-BUTANAL

2-METHYL-BUTYR-2-ALDEHYDE

2-METHYL-PENT-2-EN-1-AL

2-METHYL-PENTANAL

3,4-DIMETHYL-2,5-DIOXO-2,5-DIHYDROTHIOPHENE

3,4-DIMETHYLTHIOPHENE

31-NORCYCLOARTENOL

31-NORLANOSTENOL

4-α-METHYL-ZIMOSTENOL

5-DEHYDRO-AVENASTEROL

5-HEXYL-CYCLOPENTA-1,3-DIONE

5-METHYL-2-n-HEXYL-2,3-DIHYDROFURAN-3-ONE

5-OCTYL-CYCLOPENTA-1,3-DIONE

6(G)-β-FRUCTOSYL-SUCROSE

9,10,13-TRIHYDROXY-OCTADEC-11-ENOIC-ACID

9,12,13-TRIHYDROXY-OCTADEC-10-ENOIC-ACID

ABSCISSIC-ACID

ACETAL

ACETIC-ACID

ALANINE

ALLICIN

ALLIIN

ALLIOFUROSIDE-A

ALLIOSPIROSIDE-A

ALLIOSPIROSIDE-B

ALLIOSPIROSIDE-C

ALLIOSPIROSIDE-D

ALLYL PROPYL SULFIDE

ALLYL PROPYL TRISULFIDE

ALLYL-METHYL-DISULFIDE

ALLYL-METHYL-SULFIDE

ALLYL-METHYL-TRISULFIDE

ALLYL-PROPENYL-DISULFIDE

ALLYL-PROPYL-DISULFIDE

ALLYLTHIOL

ALUMINUM

AMMONIA

ARABINOSE

ARACHIDIC-ACID

ARGININE

ARSENIC

ASCORBIC-ACID

ASH

ASPARAGINE

ASPARTIC-ACID

BARIUM

BENZYL ISOTHIOCIANATE

BORON

BRASSICASTEROL

BROMINE

CADMIUM

CAFFEIC-ACID

CALCIUM

CALCIUM-OXALATE

CAMPHESTEROL

CARBOHYDRATES

CATECHOL

CEPAENES

CEPOSIDE-D

CHOLEST-7-EN-3-β-OL

CHOLESTEROL

CHOLINE

CHROMIUM

CIS-1-(PROPENYL-DITHIO)-PROPANE

CIS-2,3-DIMETHYL-5,6-DITHIO-CYCLO(2,2,1)HEPTANE-5-OXIDE

CIS-3,5-DIETHYL-1,2,4-TRITHOLANE

CIS-PROPANETHIOL-S-OXIDE

CIS-PROPENYL-PROPYL-DISULFIDE

CIS-PROPENYL-PROPYL-TRISULFIDE

CITOSINE

CITRIC-ACID

CITRULINE

COBALT

COPPER

CYANIDIN-3-0-LAMINARIBIOSIDE

CYANIDIN-3-0-β-D-DIGLYCOSIDE

CYANIDIN-BIOSIDE

CYANIDIN-DIGLYCOSIDE

CYANIDIN-MONOGLYCOSIDE

CYCLOALLIIN

CYCLOARTENOL

CYCLOEUCALENOL

CYSTEINE

CYSTINE

DIALLYL-DISULFIDE

DIALLYL-SULFIDE

DIALLYL-TRISULFIDE

DIHYDROALLIIN

DIISOPROPYL-TRISULFIDE

DIMETHYL-DISULFIDE

DIMETHYL-FURANE

DIMETHYL-SULFIDE

DIMETHYL-TETRASULFIDE

DIMETHYL-TRISULFIDE

DIMETHYL-TRISULFIDE

DIPHENYLAMINE

DIPROPENYL-DISULFIDE

DIPROPENYL-SULFIDE

DIPROPYL-DISULFIDE

DIPROPYL-TRISULFIDE

D-MANNITOL

EICOSEN-1-OL

EO

ETHANOL

ETHANOLAMINE

FATS

FERULIC-ACID

FIBER

FLUORINE

FRUCTOSAN

FRUCTOSE

FUMARIC-ACID

GIBERELLIN-A-4

GLUCINE

GLUCOFRUCTAN

GLUCOSE

GLUTAMINE

GLUTAN

GLYCINE

GLYCOLIC-ACID

GRAMISTEROL

HEXADECEN-1-OL

HISTIDINE

HYDROGEN SULFUR

IRON

ISOLEUCINE

ISOPROPYL-PROPYL-DISULFIDE

ISOPROPYL-PROPYL-TRISULFIDE

KAEMPFEROL

KAEMPFEROL-3,4′-DI-0-β-D-GLUCOSIDE

KAEMPFEROL4′,7-DI-0-β-D-GLUCOSIDE

KAEMPFEROL-4′-O-β-D-GLUCOSIDE

LEAD

LEUCINE

LINOLEIC-ACID

LITHYUM

LOPHENOL

LYSINE

MAGNESIUM

MALIC-ACID

MANGANESE

MERCURY

METHANOL

METHANOTHIOL

METHIONINE

METHIONINE-METHYLSULFONIUM

METHIONINE-SULFONE

METHYL-ALLIIN

METHYL-CIS-PROPENYL-DISULFIDE

METHYL-DITHIOMETHANE

METHYL-METHANOTHIOSULFONIUM

METHYL-PROPENYL-SULFIDE

METHYL-PROPENYL-TRISULFIDE

METHYL-PROPYL-DISULFIDE

METHYL-PROPYL-TRISULFIDE

METHYL-TRANS-PROPENYL-DISULFIDE

MEVALONIC-ACID

MOLYBDENUM

MUFA

MYRISTIC-ACID

MYROSINASE

NIACIN

NICKEL

NITROGEN

NONADECANOIC-ACID

N-PROPYL-MERCAPTAN

OLEANOLIC-ACID

OLEIC-ACID

OXALIC-ACID

PAEONIDIN-GLYCOSIDE

PALMITIC-ACID

PANTOTHENIC-ACID

P-COUMARIC-ACID

PECTIN

PELARGONIDIN-MONOGLYCOSIDE

PENTOSAN

PEROXIDASE

PHENILALANINE

PBLOROGLUCINOL

PHLOROGLUCYOL-CARBOXYLIC-ACID

PHOSPHORUS

P-HYDROXYBENZOIC-ACID

PHYROCATECOL

PHYTOHORMONE

PHYTOSTEROLS

PIPECOLIC-ACID

POTASSIUM

PROLINE

PROPAN-1-OL

PROPANAL

PROPANALDEHYDE

PROPANE-1-THIOL

PROP-CIS-ENYL-PROPYL-DISULFIDE

PROP-CIS-ENYL-PROPYL-TRISULFIDE

PROPENE

PROPENYL-PROPYL-SULFIDE

PROP-TRANS-ENYL-PROPYL-DISULFIDE

PROP-TRANS-ENYL-PROPYL-TRISULFIDE

PROPYL-METHANOTHIOSULFONATE

PROPYL-PROPANOTHIOSULFONATE

PROSTAGLANDIN-A-1

PROTEIN

PROTOCATECHUIC-ACID

PUFA

PYRUVIC-ACID

QUERCETIN

QUERCETIN-3,4′-DI-0-β-D-GLUCOSIDE

QUERCETIN-3-0-β-D-GLUCOSIDE

QUERCETIN-4′,7-DI-0-P-D-GLUCOSIDE

QUERCETIN-4-0-β-D-GLUCOSIDE

QUINIC-ACID

RAFFINOSE

RHAMNOSE

RIBOFLAVIN

RIBOSE

RUBIDIUM

RUTIN

S-(2-CARBOXY-PROPYL)-GLUTATHIONE

S-(β-CARBOXYBETA-METHYL-ETHER-CISTEINE

S-ALLIL-CYSTEINE

SAPONIN

SELENIUM

SELENO-CYSTEINE

SELENO-METHIONINE

SELENO-METHYLSELENOCYSTEINE

SELENO-METHYL-SELENOMETHIONINE

SELENOSIDE

SERINE

SFA

SILICON

SILVER

SINAPIC-ACID

S-METHYL-CYSTEINE

S-METHYL-CYSTEINE-SULFOXIDE

SODIUM

SPIRAEOSIDE

S-PROP-1-ENYL-CYSTEINE-S-OXIDE

S-PROPYL-1-ENYL CYSTEINESULFOXIDE

S-PROPYL-CYSTEINE-SULFOXIDE

STEARIC-ACID

STIGMAST-7-EN-3-β-OL

STIGMASTEROL

STRONTIUM

SUCCINIC-ACID

SUCROSE

SULFUR

TARTARIC-ACID

THIAMIN

THIOPROPANAL-S-OXIDE

THIOPROPIONAL-S-OXIDE

TITANIUM

TRANS-1-(PROPENYL-DITHIO)-PROPANE

TRANS-2,3-DIMETHYL-5,6-DITHIA-CYCLO-(2,2,1)-HEPTANE-5-OXIDE

TRANS-3,5-DIETHYL-1,2,4-TRITHIOLANE

TRANS-PROPENYL-PROPYL-DISULFIDE

TRANS-PROPENYL-PROPYL-TRISULFIDE

TRANS-S-(1-PROPENYL)-CYSTEINE-SULFOXIDE

TREONINE

TREDECAN-2-ONA

TRIGONELLINE

TRYPTOPHAN

TSEPOSIDES

TULIPOSIDE-A

TULIPOSIDE-B

TYROSINE

VALINE

VANILLIC-ACID

VIT-B-6

WATER

XYLITOL

XYLOSE

ZINC

ZIRCONIUM

α-AMYRIN

α-SITOSTEROL

α-TOCOPHEROL

β-ALALNINE

β-CAROTENE

β-SITOSTEROL

β-TOCOPHEROL

γ-ABULINE

γ-AMINOBUTIRIC-ACID

γ-GLUTAMYL-LEUCINE

γ-GLUTAMYL-METHIONINE

γ-GLUTAMYL-PHENYLALANINE

γ-GLUTAMYL-PHENYLALANINE-ETHYL-ESTER

γ-GLUTAMYL-S-METHYL-CYSTEINE

γ-L-GLUTAMYL-ARGININE

γ-L-GLUTAMYL-CYSTEINE

γ-L-GLUTAMYL-ISOLEUCINE

γ-L-GLUTAMYL-S-(1-PROPENYL)L-CYSTEINE-SULFOXIDE

γ-L-GLUTAMYL-S(2-CARBOXY-N-PROPYL)L-CISTEINE

γ-L-GLUTAMYL-S-(2-CARBOXY-β-METHYL-ETHYL)-CYSTEINYL-GLY

γ-L-GLUTAMYL-S-(2-CARBOXY-β-METHYL-ETHYL)-CYSTEINYL-GLY

γ-L-GLUTAMYL-VALINE

CYANIDIN-3-MALONYLGLUCOSIDE

CYANIDIN-3-MANOLYLAMINARIBIOSIDE

PEONIDIN-3-GLUCOSIDE

PEONIDIN-3-MALONYLGLUCOSIDE

PHOSPHATASE

PROPILENSULFIDE

QUERCETIN-3,4′-O-β-DIGLUCOPYAOSIDE

QUERCETIN-3,7,4′-O-β-GLUCOPYRANOSIDE

QUERCETIN-4′-O-β-GLUCOPYRANOSIDE

TAXIFOLIN-4′-O-b-GLUCOPYRANOSIDE

β-FRUCTOFURANOSIDASE

γ-GLUTAMYLTRANSPEPTIDASE

Of the above materials, those that are especially hypothesized to be responsible for the medicinal and immune regulating activity of the subject Allium extract are set forth below.

1-O-CAFFEOYL-b-D-GLUCOSE

1-O-P-COUMAROYL-b-D-GLUCOSE

ALLICIN

ALLIIN

ALLIOFURÓSIDE-A

ALLIOSPIRÓSlDE-A

ALLIOSPIRÓSIDE-B

ALLIOSPIRÓSIDE-C

ALLIOSPIRÓSIDE-D

ALLYL PROPYL SULFIDE

ALLYL PROPYL TRISULFIDE

ALLYL-METHYL-DISULFIDE

ALLYL-METHYL-SULFIDE

ALLYL-METHYL-TRISULFIDE

ALLYL-PROPENYL-DISULFIDE

ALLYL-PROPYL-DISULFIDE

CAFFÉIC-ACID

CAMPHESTEROL

CATECHOL

CHOLINE

CIS-PROPENYL PROPYL-DISULFIDE

CIS-PROPENYL-PROPYL-TRISULFIDE

CYANIDIN-3-O-LAMINARIBIÓSIDE

CYANEDIN-3-O-b-D-DIGLYCÓSIDE

CYANIDIN-BIÓSIDE

CYANIDIN-DIGLYCÓSIDE

CYANIDIN-MONOGLYCÓSIDE

CYCLOALLIIN

DIALLYL-DISULFIDE

DIALLYL-SULFIDE

DIALLYL-TRISULFIDE

DIHYDROALLIIN

DIISOPROPYL-TRISULFIDE

DIMETHYL-DISULFIDE

DIMETHYL-SULFIDE

DIMETHYL-TETRASULFIDE

DIMETHYL-TRISULFIDE

DIPROPENYL-DISULFIDE

DIPROPENYL-SULFIDE

DIEPROPYL-DISULFIDE

DIPROPYL-TRISULFIDE

HYDRÓGEN SULFUR

ISOPROPYL-PROPYL-DISULFIDE

ISOPROPYL-PROPYL-TRISULFIDE

KAEMPFEROL-3,4′-DI-O-b-D-GLUCOSIDE

KAEMPFEROL-4′,7-DI-O-b-D-GLUCOSIDE

KAEMPFEROL4′-O-b-D-GLUCOSIDE

LYSINE

METHIONINE-METHYLSULFONIUM

METHIONINE-SULFONE

METHYL-ALLIIN

METHYL-CIS-PROPENYL-DISULFIDE

METHYL-METHANOTHIOSULFONIUM

METHYL-PROPENYL-SULFIDE

METHYL-PROPENYL-TRISULFIDE

METHYL-PROPYL-DISULFUIDE

METHYL-PROPYL-TRISULFIDE

METHYL-TRANS-PROPENYL-DISULFIDE

MYRÍSTIC-ACID

MYROSINASE

OLEANÓLIC-ACID

PAEONIDIN-GLYCOSIDE

PALMÍTIC-ACID

P-COUMÁRIC-ACID

PELARGONIDIN-MONOGLYCOSIDE

PROP-CIS-ENYL-PROPYL-DISULFIDE

PROP-CIS-ENYL-PROPYL-TRISULFIDE

PROPENYL-PROPYL-SULFIDE

PROP-TRANS-ENYL-PROPYL-DISULFIDE

PROP-TRANS-ENYL-PROPYL-TRISULFIDE

PROPYL-METHANOTHIOSULFONATE

PROPYL-PROPANOTHIOSULFONATE

PROSTAGLANDIN-A-1

QUERCETIN

QUERCETIN-3,4′-DI-O-b-D-GLUCÓSIDE

QUERCETIN-3-O-b-D-GLUCÓSIDE

QUERCETIN-4′,7-DI-O-b-D-GLUCÓSIDE

QUERCETIN-4-O-b-D-GLUCÓSIDE

RUTIN

S-ALLIL-CYSTEINE

SAPONIN

SELENIUM

SELENO-CYSTEINE

SELENO-METHIONINE

SELENO-METHYLSELENOCYSTEINE

SELENO-METHYL-SELENOMETHIONINE

SELENÓSEDE

S-METHYL-CYSTEINE-SULFÓXIDE

S-PROPYL ENYL-1-ENYL CYSTEINESULFÓXIDE

S-PROPYL-CYSTEINE-SULFÓXIDE

SULFUR

TRANS-PROPENYL-PROPYL-DISULFIDE

TRANS-PROPENYL-PROPYL-TRISULFIDE

TRANS-S-(1-PROPENYL)-CYSTEINE-SULFÓXIDE

a-AMYRIN

a-SITOSTEROL

b-SITOSTEROL

g-GLUTAMYL-LEUCINE

g-GLUTAMYL-METHIONINE

g-GLUTAMYL-PHENYLALANINE

g-GLUTAMYL-PHENYLALANINE-ETHYL-ESTER

g-GLUTAMYL-S-METHYL-CYSTEINE

g-L-GLUTAMYL-ARGININE

g-L-GLUTAMYL-CYSTEINE

g-L-GLUTAMYL-ISOLEUCINE

g-L-GLUTAMYL-S-(1-PROPENYL)L-CYSTEINE-SULFÓXIDE

g-L-GLUTAMYL-S(2-CARBOXY-N-PROPYL)1-CISTEINE

g-1,GLUTAMYL-S-(2-CARBOXY-b-METHYL-ETHYL)-CYSTEINYL-GLY

g-L-GLUTAMYL-VALINE

CYANIDIN-3-MALONYLGLUCÓSIDE

CYANIDIN-3-MANOLYLAMINARIBIOSIDE

QUERCETIN-3,4′-O-b-DIGLUCOPYRANOSIDE

QUERCETIN-3,7,4′-O-b-GLUCOPYRANOSIDE

QUERCETIN-4′-O-b-GLUCOPYRANOSIDE

Of these compounds, the following are believed to be the most likely candidates to be separately or jointly responsible for the medicinal properties of the subject Allium extracts.

1-O-CAFFEOYL-b-D-GLUCOSE

ALLICIN

ALLIIN

ALLYL PROPYL SULFIDE

ALLYL PROPYL TRISULFIDE

ALLYL-METHYL-DISULFIDE

ALLYL-METHYL-SULFIDE

ALLYL-METHYL-TRISULFIDE

ALLYL-PROPENYL-DISULFIDE

ALLYL-PROPYL-DISULFIDE

CAFFÉIC-ACID

CATECHOL

CHOLINE

DIHYDROALLIN

KAEMPFEROL-3,4′-DI-O-b-D-GLUCOSIDE

KAEMPFEROL-4′,7-DI-O-b-D-GLUCOSIDE

KAEMPFEROL-4′-O-b-D-GLUCOSIDE

LYSINE

METHYL-METHANOTHIOSULFONIUM

OLEANÓLIC-ACID

PALMÍTIC-ACID

P-COUMÁRIC-ACID

PROPYL-METHANOTHIOSULFONATE

PROPYL-PROPANOTHIOSULFONATE

QUERCETIN

QUERCETIN-3,4′-DI-O-b-D-GLUCÓSIDE

QUERCETIN-3-O-b-D-GLUCÓSIDE

QUERCETIN-4′,7-DI-O-b-D-GLUCÓSIDE

QUERCETIN-4-O-b-D-GLUCOSIDE

RUTIN

SAPONIN

SELENIUM

a-AMYRIN

a-SITOSTEROL

b-SITOSTEROL

CYANIDIN-3-MALONYLGLUCÓSIDE

CYANIDIN-3-MANOLYLAMINARIBIOSIDE

QUERCETIN-3,4′-O-b-DIGLUCOPYRANOSIDE

QUERCETIN-3,7,4′-O-b-GLUCOPYRANOSIDE

QUERCETIN-4′-O-b-GLUCOPYRANOSIDE

However, Applicants do not want to be bound by such hypotheses. In fact, it may be that the medicinal properties of the subject Allium extracts may involve different constituents, or may require a particular distribution of constituents that are selectively obtained by the afore-described processing procedure. Moreover, as discussed previously, the particulate size or other morphological properties of the material may also be significant to medicament activity.

The particles obtained by the described procedure may be administered by systemic or non-systemic means. Typically, the particles (in the form of a powder) will be placed in capsules that either dissolve in the stomach or intestine, or both, or will be used to make tablets, suppositories, sachets, or will liquid administrable forms, e.g., elixirs, syrups, or suspensions. Alternatively, these powders can be used to produce an injectable composition, e.g., by addition to a pharmaceutically acceptable excipient such as buffered saline. Of these modes of administration, oral administration is preferred. For example, in a preferred embodiment, capsules containing the powder according to the invention may be ingested with an ingestible fluid, e.g., juice, water, or milk. Still alternatively, the powders will be added to a food, e.g. solid or liquid that camouflages the taste of the particles, especially if the user does not like the taste of onion.

Methods for producing orally administrable materials having desired properties, e.g., sustained or rapid release, enteric-coated forms, are well known and are described in Remington's Pharmaceutical Sciences, Mack Publishers (incorporated by reference herein.)

In producing capsules, the subject Allium powder may be combined with other materials, if desired, e.g, sugars such as lactose, sucrose, mannitol, starches, cellulose derivatives, magnesium stearate or stearic acid. Also, materials that enhance aesthetic properties of the material may be added, e.g., colorants and flavoring materials. Additives which can be utilized in capsule formulations are well known to those skilled in the art.

If the subject powders are utilized to make tablets, conventional tableting procedures can be used which typically comprise processing the materials by compression to produce a tablet. Materials which facilitate tablet formation can be utilized, e.g., binders and bulking agents or other additives, including by way of example gums, waxes, insoluble polymers, polyvinyl alcohol, polyethylene glycol, sucrose, lactose, acacin, tragacanth, and polyvinyl pyrrolidine. Also, additives may be utilized which enhance taste and appearance, e.g., flavoring or coloring agents.

In a preferred embodiment, the tablets are coated such that they are selectively released in the stomach or intestine. With respect thereto, coatings which are acid-stable and allow for drug release in the intestine (referred to generally as enteric coatings) are well known. Examples include shellac and derivatives thereof, cellulose acetate phthalate, hydroxypropyl/methylcellulose phthalate, ethyl cellulose. Such enteric coating and methods for application thereof are discussed in detail in Remington's Pharmaceutical Sciences, (Id.) Also, U.S. Pat. Nos 4,017,647 and 4,287,221 are exemplary of enteric coated drug formulations. The enteric coated form should enhance the antimicrobial properties of the powder.

Alternatively, and preferably in the case of subjects that find solid dosage formulations difficult to take (which may be a significant concern in AIDS patients who at latter stages may have swallowing problems), the subject materials may be produced in the form of liquid elixirs, or other liquids. This can be effected by combining the subject Allium powder with fruit or other vegetable juices, sugars, flavoring materials, or other materials having known application in liquid drug formulations. This is further advantageous for subjects that find the taste of the subject material disagreeable. Still alternatively, the subject material may be combined with solid foods, e.g., in order to camouflage the taste, if desired.

As noted, a less preferred means of administration will comprise injectable formulations. In this embodiment, the powder will be combined with an injectable liquid, e.g., buffered saline, and injected by known routes, e.g., intravenous, intramuscular, intradermal, or subcutaneous routes of administration. For example, a subject with an AIDS-associated lesion may be injected at the site of lesion to elicit an immune modulating or T-cell response.

Other modes of administration include topical, inhalatory, intranasal, sustained release implants, and rectal or vaginal suppositories.

The amount of particles which are administered, e.g., orally, injection, suppository, intranasal, will typically range from about 5-50 grams per day, and more preferably about 9 to 13 grams per day. A particular advantage of the medicament of the present invention is that there are no known side effects. Consequently, there are no real upper dosage ranges.

A general description of properties of the subject material is summarized below.

1. Description of the Medicinal Allium Product of the Invention

Properties of the medicinal product of the invention were evaluated by placing 10 grams of this material in 500 ml of boiled water and heated for 10 minutes. The rehydrated vegetable exhibited the following characteristics:

| Look & Color | Flavor | Aroma |
|---|---|---|
| White flakes with some green particles | Characteristic | Characteristic |

2. Granulometric

| | | |
|---|---|---|
| Retained on sieve | USA 3/8 (9.5 mm) | 5% max |
| Retained on sieve | USA 8 (2.36 mm) | 99% min |
| Through sieve | USA 8 | 5% max |

3. Humidity

Max 5.5%

4. Microbiological Analysis

| | | | |
|---|---|---|---|
| Total Aerobic Mesophiles | 300,000 | ufc/g | Maximum |
| Total Coliforms | 1,000 | NMP/g | Maximum |
| E. coli | Negative | | |
| Reduce sulphite Clostridium | 10 | ufc/g | Maximum |
| Yeast and Molds | 1,000 | ufc/g | Maximum |

As noted above, the subject materials can be stored for prolonged periods prior to usage, preferably by storing in a dry, ambient temperature conditions, i.e., about 18-25° C., preferably in the dark to avoid oxidation. Preferably, the material will be stored in a sealed plastic bag or other container to maintain low humidity and avoid microbial or other contamination.

As discussed previously, the subject medicament has a number of different properties that render it well suited for use as a therapeutic. For example, it has been found that the subject Allium derived medicament has broad antiviral activity. The subject extract may be used to treat or prevent a variety of different viral infections, both human and animal viruses. Examples thereof include retroviral infections such as AIDS, herpes (genital, rectal, oral), distemper, papillomavirus, flu associated influenza viruses, parvoviruses, rhabdoviruses, Epstein Barr virus, CMV, hepatitis virus, RSV, rhinoviruses, and foot and mouth disease virus.

In the preferred embodiment, the subject Allium derived medicament will be used for the treatment of AIDS. Entirely unexpectedly, it has been found that administration of the subject medicament to patients with AIDS results in disappearance of elimination of the clinical symptoms associated therewith, such as wasting syndrome, paresthesia intestinal colic, diarrhea, polyadenopathy, and HIV related infections. To date, these results have been observed in eight different patients having an age ranging from 28 to 38, many of which were in the latter terminal (“C-stage”) of AIDS. These clinical results are summarized in the example infra.

In effecting the HIV treatment protocol of the invention, patients with AIDS will be withdrawn~from anti-AIDS conventional medications such as protease inhibitors, anti-retrovirals, cytotoxic drugs, steroids, chemotherapies, and preferably will be placed on a restricted diet developed by the inventors. The use of tobacco and other smoking products or narcotics is also forbidden.

This diet preferably comprises ingestion of only natural products (without preservatives, and chemical additives) such as vegetables, fruits, fish, meat in small quantities, no fried foods, no alcoholic beverages, no caffeinated beverages such as coffee, tea, and no sweetened drinks such as sodas. The use of tobacco and other smoking products, or narcotics, is also forbidden. If intestinal irregularities result because of ingestion of large amounts of vegetables and fruit, this preferably will be treated by ingestion of rice, white bread, cheese, apples or lemon. However, if necessary an anti-diarrheic medicine may be administered. Moreover, if significant weight loss results, then unfried potatoes, preferably about 250 grams twice a day may be ingested.

The subject AIDS therapy will comprise administration, preferably about 9-13 g/day of the subject medicament. Preferably, this will be effected orally, e.g., in capsule form by mixing in a suitable beverage or with food. However, other known modes of administration can be used. This treatment should be continued for the life of the patient. A maintenance dose is established once the patient is free of symptoms. This maintenance dosage typically will range from about 5-7 g/day. In fact, it has been observed for some patients who have stopped this treatment, that AIDS symptoms have relapsed. However, when these patients resumed the subject treatment, remission (absence of clinical symptoms) again resulted. Ideally, the treated patients will also undergo an exercise regimen/regime to help enhance overall wellness.

Also, the subject medicament may be used to treat other conditions. In particular, the subject medicament may be used to modulate the immune system, stimulate the immune system, and/or enhance T cell function and/or proliferation in subjects in need of such treatment. Examples where such treatment will be of therapeutic benefit include boosting the immune systems of aged or immunosuppressed persons, persons with cancer, and persons with infection. In particular, the subject medicament has application in treatment and/or prevention of microbial infection, e.g., by fungi.

Still further, the subject medicament may be used to induce weight gain in persons in need of such treatment, e.g., those suffering from anorexia.

These treatments will be effected substantially the same as AIDS treatment, i.e., an effective amount of the subject Allium cepa material will be administered, preferably by oral administration, typically a daily dosage of about 9-13 g/day or more.

Also, the subject medicament may be used to treat infections, e.g., viral or bacterial infections, in animals such as dogs, cats, and birds. In fact, the means by which the subject medicament was initially shown to be effective involved treatment of a dog with parvovirus infection. As with AIDS treatment, this treatment has been observed to totally eradicate the clinical symptoms of the disease in the treated animal. Other animal diseases which have been treated include distemper, and psittacosis.

Still other diseases and conditions treatable with the subject Allium extract include candidiasis, e.g. pneumonia, caused by Pneumocistis carinii, urinary infection, and mycosis.

### EXAMPLE

Allium cepa extracts produced according to the invention were orally administered to eight persons that are HIV⁺, most of them with full blown AIDS. In fact, some of these persons were close to death when treatment initiated. Treatment comprised stopping conventional treatment, starting of the dietary regimen according to the invention, and oral ingestion of about 9 to 13 g/day of the subject Allium extract daily. As can be seen from the results in the Table below, dramatic results were achieved, i.e., all of these eight persons had a total remission of clinical symptoms associated with AIDS and were able to resume a normal life style after treatment.

CLINICAL CHARACTERISTICS OF PATIENTS WITH HIV INFECTION TREATED WITH IMMUNO-PLUS AND DIETARY REGIMEN

| PATIENT No. | 103* | 105 | 101 | 109 | 108 | 106* | 107* | 102* |
|---|---|---|---|---|---|---|---|---|
| AGE | 38 | 28 | 32 | 34 | 29 | 31 | 30 | 30 |
| SEX | M | M | M | M | M | M | M | M |
| TE HIV DIAGNOSIS | 1991 | 1994 | 1994 | 1993 | 1993 | 1996 | 1994 | 1997 |
| CDC CATEGORY | C.2 | C.2 | C.2 | B 1 | A.1 | C.1 | B 1 | C.3 |
| STARTING DATE | Sep. 1, 1997 | Jun. 26, 1997 | Jun. 24, 1997 | Jun. 4, 1993 | Oct. 6, 1993 | Mar. 25, 1996 | Jan. 28, 1994 | Nov. 10, 1997 |
| PREVIOUS | ANTIBIOTICS | ANTIBIOTICS, | NO | NO | NO | NO | ANTIBIOTICS | SUSPENDED |
| MEDICATION | (BACTRIM) | ANTIMICOTICS, | | | | | (BACTRIM) | ANTIRETROVIRALS |
| | | ANTACIDS | | | | | | 8 DAYS AGO |
| ADENOPATHY | INGUINIAL | NO | OCCIPITAL, | POLYADE- | NO | ADENOPATHY, | CERVICAL- | POLYADENOPATHTY |
| | BILATERIAL | | CERVICAL | NOPATHY | | CERVICAL, ARMPIT | ARMPIT | |
| | LEFT ARMPIT | | | | | | BILATERAL | |
| CONSTITUTIONAL | <WEIGHT, | 39° C. | <WEIGHT, <7 KG 90 | <WEIGHT | NO | <4 KG-6 MO. | <4 KG-CONT. | <18 KG-6 MONTHS |
| SYNDROME | INTESTINAL | | DAYS, INTERMIT. UP | FEVER | | INTERMIT. DIARRHEA, | DIARRHEA, | CONT DIARRHEA, |
| | DIARRHEA, | | TO 38° C. | | | FEVER | FEVER | FEVER 38° C. |
| | 38° C. PERSISTENT | | | | | | | |
| HISTORY OF HIV | ORAL/RECTAL | ORAL HERPES, ORAL | ORAL HERPES, | NO | NO | PNEUMONIAS | PNEUMONIAS | RECTAL HERPES, |
| RATED INFECTIONS | HERPES, | MICOSIS, PNEUMONIAS | PNEUMONIA | | | | | SIGMOIDITIS, |
| | OR OF ARINGEAL | | | | | | | PNEUMONIAS, |
| | MICOSIS, | | | | | | | URINARY |
| | PNEUMONIA | | | | | | | INFECTIONS |
| GASTRO-INTESTINAL | EPIGASTRALGIA, | NAUSEA, ANOREXIA, | INTERMIT. | NO | NO | ANOREXIA | ANOREXIA | INTESTINAL COLICS, |
| SYMPTOMS | ANOREXIA, | INTESTINAL COLICS | DIARRHEA, | | | | | INTESTINAL |
| | DIARRHEA, | | INTESTINAL COLICS | | | | | PERFORATION AND |
| | INTESTINAL | | | | | | | SURGERY ON Oct. 1997 |
| | COLICS | | | | | | | |
| OTHER CLINICAL | PARESTHESIAS OF | SEVERE SHORTNESS | DEPRESSION, | DEPRESSION, | NO | GASTRITIS, ANOREXIA | TREATED IN — | OPENED INFECTED |
| ASPECTS | LOWER LIMBS, | OF BREATH, OXYGEN | GENERAL | SUBMAXIL- | | | BUENOS AIRES | SURGERY WOUND, |
| | DEPRESSION, | THERAPY, RESERVED | DISCOMFORT, | LARY | | | FOR | EMACIATION, |
| | RIGHT ORCHITIS | PROGNOSIS | ANXIETY | ABSCESS | | | PNEUMONIAS | DYSPNEA III-IV, |
| | | | | | | | | TERMINAL PATIENT |
| EVOLUTION | GAINED 4 KG, NO | <WEIGHT, PHYSICAL | PHYSICAL | PHYSICAL | PHYSICAL | PHYSICAL EXAMINATION | PHYSICAL | >14 KG TO DATE, |
| JAN-FEB '98 | INFECTIOUS SYMP- | EXAMINATION | EXAMINATION | EXAMINA- | EXAMINA- | NORMAL | EXAMINATION | PALENESS, |
| | TOMATOLOGY, | NORMAL | NORMAL | TION | TION | | NORMAL | WEAKNESS, GOOD |
| | GOOD GENERAL | | | NORMAL | NORMAL | | | PROGRESS |
| | CONDITION | | | | | | | |

| | |
|---|---|
| EVOLUTION | ALL PATIENTS ARE CURRENTLY IN GOOD CONDITION, PHYSICAL EXAM NORMAL (**) |
| DECEMBER '99 | |

PNEUMONIAS: 80% PNEUMONIAS WITH PNEUMOCISTIS CARINII (PCP)
20% PNEUMONIAS W/COMMON GERMS (COCOS)
*RECEIVED ANTI-RETROVIRALS UNTIL STARTING WITH I+ (Allium cepa extract according to the invention)
** PHYSICAL EXAMINATION NORMAL
VITAL SIGNS WITHIN NORMAL LIMITS (BP, PULSE, TEMPERATURE IN DEGREES CELSIUS AND RESPIRATORY RATE)
NO WEIGHT LOSS
NO ADENOPATHIES
NO DIARRHEA
NO AIDS RELATED INFECTIONS
RESPIRATORY, CARDIO-VASCULAR, ABDOMINAL, GENITO-URINARY, RECTAL AND NEUROLOGICAL EXAMS, ALL NORMAL

What is claimed is:

1. A method for treating an HIV positive or AIDS patient comprising administering a therapeutically effective amount of a plant extract derived from an Allium species other than A. sativum, wherein said plant extract is obtained from a dehydrated Allium plant material that is processed after dehydration to produce particles having an average particle size ranging from about 1 to 1,400 microns.

2. The method of claim 1, wherein said Allium plant extract is administered orally.

3. The method of claim 1, wherein said Allium plant extract is produced from an Allium selected from the group consisting of A. cepa, A. ampeloprasum, A. fistulosa, and A. schoenoprasum.

4. The method of claim 3, wherein said Allium is A. cepa.

5. The method of claim 1, which comprises daily administration of about 1 to 50 grams of said particulate Allium plant extract.

6. The method of claim 1, which lengthens the latent phase of AIDS.

7. The method of claim 1, which results in alleviation, improvement, or eradication of wasting syndrome, or other clinical symptoms associated with AIDS or HIV⁺ patients.

8. A method for treating or preventing viral infection in a patient in need of such treatment or prevention comprising administering a therapeutically or prophylactically effective amount of a plant extract is obtained from a dehydrated Allium plant material that is processed after dehydration to produce particles having an average particle size ranging from about 1 to 1,400 microns.

9. The method of claim 8, wherein said Allium extract is administered orally.

10. The method of claim 8, wherein said viral infection is selected from the group consisting of influenza, herpes, hepatitis, parvovirus, distemper, RSV, CMV, rhinovirus, rhabdovirus, papillomavirus, Epstein Barr, and foot and mouth disease virus.

11. The method of claim 8, wherein said Allium is selected from the group consisting of A. cepa, A. ampeloprasum, A. fistulosa, and A. schoenoprasum.

12. The method of claim 9, wherein said Allium is A. cepa.

13. The method of claim 8, wherein said particulate extract is administered orally.

14. The method of claim 1, wherein said particulate extract is administered orally.

15. A method for promoting appetite and/or weight gain in a patient in need of such treatment, comprising administering an effective amount of a plant extract derived from an Allium species other than A. sativum, wherein said plant extract is obtained from a dehydrated Allium plant material that is processed after dehydration to produce particles having an average particle size ranging from about 1 to 1,400 microns.

16. A method for stimulating and/or modulating the immune system of a subject in need of such treatment comprising administering an immunostimulating and/or immuno-modulating effect amount of a plant extract derived from an Allium species other than A. sativum, which extract is obtained from a dehydrated Allium plant material that is processed after dehydration to produce particles having an average particle size ranging from about 1 to 1,400 microns.

17. The method of claim 1, which is used to boost the immune system of a subject having an immune system that is compromised by age, disease, and/or inadequate nutrition.

18. The method of claim 16, wherein said Allium is-selected from the group consisting of A. cepa, A. ampeloprasum, A. fistulosa, and A. schoenoprasum.

19. A method for enhancing T-cell function, proliferation and/or differentiation in a subject in need of such treatment comprising administering an effective amount of a plant extract derived from an Allium species other than A. sativum, wherein said extract is obtained from a dehydrated Allium plant material that is processed after dehydration to produce particles having an average particle size ranging from about 1 to 1,400 microns.

20. A method for treating microbial infection in a subject in need of such treatment comprising administering an effective amount of a plant extract derived from an Allium species other than A. sativum, which extract is obtained from a dehydrated Allium plant material that is processed after dehydration to produce particles having an average particle size ranging from about 1 to 1,400 microns.

21. The method of claim 20, which is used to treat yeast infection.

22. The method of claim 20, which is used to treat fungal infection.

23. The method of claim 20, wherein said Allium is selected from the group consisting of A. cepa, A. ampeloprasum, A. fistulosa, and A. schoenoprasum.

24. The method of claim 23, wherein said Allium extract is derived from A. cepa.

25. The method of claim 1, which further comprises placing said patient on a restricted diet.

26. The method of claim 1, wherein said patient is not being treated with any other anti-AIDS therapeutics.

27. A method for producing an Allium plant extract having antiviral, antimicrobial and/or immuno-modulating properties comprising the following steps:
(1) obtaining an Allium plant material which includes at least the bulb portion of the plant, wherein said Allium is of a species other than A. sativum;
(2) subjecting said plant material to one or more washing procedures, wherein washing is effected using a chlorinated aqueous solution;
(3) dehydrating said washed Allium plant material by heating at a temperature ranging from about 80° to 110° C.; and
(4) processing said dehydrated Allium plant material under low humidity to produce a composition comprised of particles wherein the average particle size ranges from about 1 to 1,400 microns.

28. The method of claim 27, wherein the washed plant material is cut into thin slices prior to dehydration.

29. The method of claim 27, wherein the particulate composition resulting from step (4) is placed into capsules.

30. The method of claim 27, wherein the particulate composition resulting from step (4) is used to produce suppositories tablets, or sachets.

31. The method of claim 27, wherein the particulate composition resulting from step (4) is used to produce a liquid orally administrable formulation.

32. The method of claim 29, wherein said tablets are coated such that they dissolve selectively in the stomach or intestine, or comprise a mixture of coated tablets that dissolve selectively in the stomach and intestine.

33. A medicinal extract derived from an Allium species other than A. sativum produced according to claim 27.

34. The medicinal extract of claim 33, which is obtained from A. cepa.

35. The medicinal extract of claim 33, which is in the form of a powder, capsule, tablet suppository, sachet, injectable composition, oral administrable liquid, inhalatory, aerosol, or topically administrable composition.

36. The medicinal extract of claim 35, which comprises a tablet.

37. The medicinal extract of claim 35, which comprises a capsule.

38. The medicinal extract of claim 35, which comprises a suppository.
